(19) United States
(12) Patent Application Publication
(10) Pub. No.: US 2026/0324988 A1
(43) Pub. Date: Oct. 22, 2026

(54) Passive Proximity Detection using an Electrode

(71) Applicant: Google LLC, Mountain View, CA (US)

(72) Inventors: Ke-Yu Chen, San Ramon, CA (US); Debanjan Mukherjee, Palo Alto, CA (US); Xiaoyu Guo, Santa Clara, CA (US); Hong Z. Tan, Redwood City, CA (US); Samuel Wei Sheng, Saratoga, CA (US); Kevin Drake, Campbell, CA (US)

(73) Assignee: Google LLC, Mountain View, CA (US)

(21) Appl. No.: 19/117,379

(22) PCT Filed: Sep. 30, 2024
(86) PCT No.: PCT/US2024/049305

§ 371 Date: Mar. 31, 2025

Related U.S. Application Data
(60) Provisional application No. 63/587,519, filed on Oct. 3, 2023.

(51) Int. Cl. A61B 5/0205; A61B 5/00; A61B 5/024; A61B 5/11; A61B 5/332; G01D 5/12

(52) U.S. Cl. CPC A61B 5/0205; A61B 5/02438; A61B 5/1126; A61B 5/332; A61B 5/4815; A61B 5/6898; G01D 5/12; A61B 2562/0257

(57) ABSTRACT

Techniques and apparatuses are described that implement passive proximity detection using an electrode. In example aspects, a computing device includes at least one electrode, which is capable of passively sensing an electric field in an external environment. By using the electrode to detect changes in the electric field, the computing device can perform proximity detection to detect the presence (or absence) of a body. This form of proximity detection can consume significantly less power compared to other active sensing techniques. The electrode can also be cheaper and/or have a smaller footprint compared to other types of sensors. Other advantages of the electrode include it being orientation agnostic, able to operate in all lighting conditions, and capable of penetrating obstructions. With the use of the electrode, proximity detection can be performed while preserving the privacy of a user.

### BACKGROUND

Electronic devices can provide a variety of features and functionality that make everyday life easier for users. Some features, however, can strain the electronic device's power resources, thereby reducing a duration of mobile operation or causing the electronic device to be recharged more often. As limitations on available power can significantly impact the utilization of electronic devices, there is an increased demand for implementing features that consume less power and minimize overall power consumption of the electronic device.

### SUMMARY

Techniques and apparatuses are described that implement passive proximity detection using an electrode. In example aspects, a computing device includes at least one electrode, which is capable of sensing an electric field in an external environment. By using the electrode to detect changes in the electric field, the computing device can perform passive proximity detection to detect the presence (or absence) of a body. This form of passive proximity detection can consume significantly less power compared to other active sensing techniques. As such, passive proximity detection using the electrode can be performed in a continuous manner to enable the computing device to dynamically respond to the body's presence or absence. The electrode can also be cheaper and/or have a smaller footprint compared to other types of proximity sensors. Other advantages of the electrode include it being orientation agnostic, able to operate in all lighting conditions, and capable of penetrating obstructions. Furthermore, with the use of the electrode, passive proximity detection can be performed while preserving a user's privacy.

Aspects described below include a method performed by computing device for performing passive proximity detection using an electrode. The method includes passively sensing, using at least one electrode of the computing device, an electric field that exists within an external environment. The method also includes detecting, based on the passive sensing, a change in the electric field. The method additionally includes detecting from the change in the electric field that a body is within a proximity range from the computing device.

Aspects described below also include an apparatus comprising at least one electrode and configured to perform any of the described methods.

Aspects described below include a computer-readable storage medium comprising computer-executable instructions that, responsive to execution by a processor, cause a computing device to perform any one of the described methods.

Aspects described below also include a system capable of performing passive proximity detection using an electrode.

### BRIEF DESCRIPTION OF DRAWINGS

Apparatuses and techniques for passive proximity detection using an electrode are described with reference to the following drawings. The same numbers are used throughout the drawings to reference like features and components:

FIG. 1 illustrates an example environment in which passive proximity detection using an electrode can be implemented;

FIG. 2 illustrates example components of a computing device for implementing aspects of passive proximity detection using an electrode;

FIG. 3 illustrates an example operation of an electrode performing passive proximity detection;

FIG. 4 illustrates an example implementation of a computing device that is capable of performing passive proximity detection using an electrode;

FIG. 5 illustrates an example scheme performed by a computing device;

FIG. 6 illustrates example data generated by an electrode for passive sensing;

FIG. 7 illustrates an example plot of signal attenuation over angle of incidence for passive sensing;

FIG. 8 illustrates an example method for performing an aspect of passive proximity detection using an electrode; and

FIG. 9 illustrates an example computing system embodying, or in which techniques may be implemented that enable use of, passive proximity detection using an electrode.

### DETAILED DESCRIPTION

Limitations on available power can significantly impact features or capabilities of an electronic device. Advance features may strain the electronic device's power resources, thereby reducing a duration of mobile operation or causing the electronic device to be recharged more often. Some power-saving techniques may turn off components of the electronic device until a user physically touches the electronic device. However, a delay associated with waiting for the user to touch the electronic device may cause the electronic device to respond slower to the user. Furthermore, there may be situations in which the user engages with the electronic device without physically touching the electronic device. An example situation can include the user watching entertainment that is presented on the electronic device and setting the electronic device on a surface so that they do not have to physically hold the device. It can be annoying to the user to have to physically touch the electronic device periodically in order to keep the display on. In general, there is a trade-off between conserving power and providing responsiveness and/or improving the user experience.

To address this issue, some electronic devices perform proximity sensing using a sensor or a component of the electronic device. By sensing a proximity of a user, an electronic device can automatically reduce power consumption when the user is not present. The electronic device can also utilize proximity sensing to automatically turn on features when the user is present, thereby improving responsiveness. With proximity sensing, the electronic device can further improve the user experience by preventing features from turning off while the user is within a vicinity of the electronic device.

Some electronic devices perform active proximity sensing through optical sensing via a camera and/or through radar sensing via a radar sensor. Cameras, however, can consume a significant amount of power, can be bulky and expensive to integrate within the electronic device, and can raise privacy concerns. A camera is also sensitive to an amount of available light, and cannot operate in the dark. While radar sensors can consume less power than cameras and can address privacy concerns, operations of some radar sensors may be subjected to restrictions such as the maximum permitted exposure (MPE) limit as determined by the Federal Communications Commission (FCC). These restrictions can limit the capabilities of the radar sensors. Both the camera and/or the radar sensor can also have a limited viewing angle. This means that providing 360 degrees of proximity detection may require multiple cameras and/or multiple radar sensors to be integrated within the electronic device.

Other electronic devices can perform passive proximity detection through wireless-local-area-network (WLAN) sensing (or Wi-Fi® sensing) using a transceiver. Wireless-local-area-network sensing, however, can be challenging to implement as it requires multiple transmitters, which makes it hard to scale to a home setting. The wireless local-area-network sensing can also be obstructed by objects within a home.

To address this challenge, techniques are described that implement passive proximity detection using an electrode. In example aspects, a computing device includes at least one electrode, which is capable of sensing an electric field in an external environment. By using the electrode to detect changes in the electric field, the computing device can perform passive proximity detection to detect the presence (or absence) of a body. This form of passive proximity detection can consume significantly less power compared to other active sensing techniques. As such, passive proximity detection using the electrode can be performed in a continuous manner to enable the computing device to dynamically respond to the body's presence or absence. The electrode can also be cheaper and/or have a smaller footprint compared to other types of proximity sensors. Other advantages of the electrode include it being orientation agnostic, able to operate in all lighting conditions, and capable of penetrating obstructions. With the use of the electrode, passive proximity detection can be performed while preserving a user's privacy.

### Operating Environment

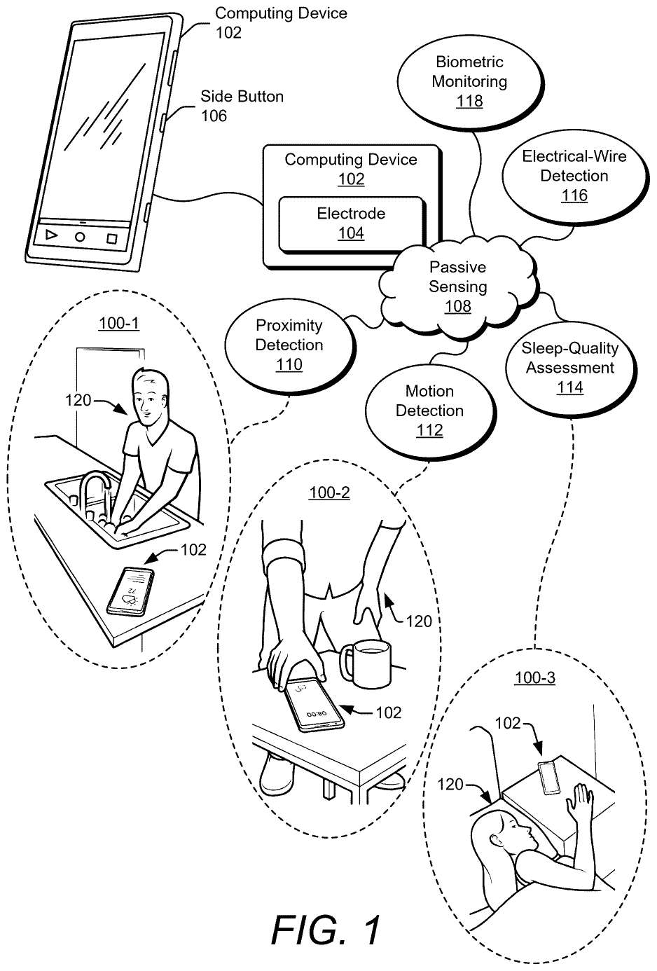

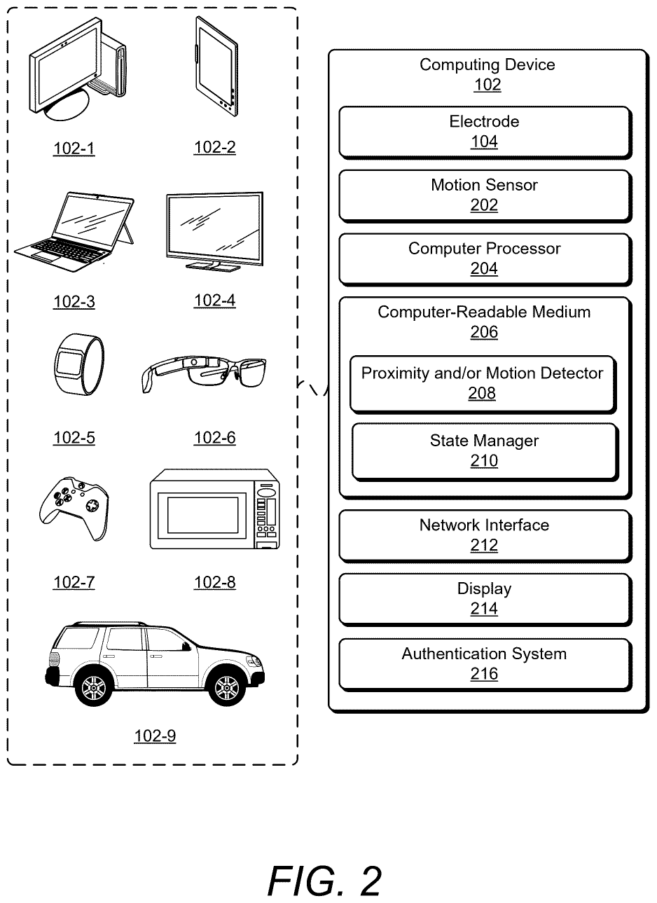

FIG. 1 is an illustration of example environments in which passive sensing using an electrode can be implemented. Consider an example computing device 102 with an electrode 104. The electrode 104 can be embedded or integrated within the computing device 102. In this case, the electrode 104 can be considered part of the computing device 102. In an example implementation, the electrode 104 is incorporated in a side button 106 of the computing device 102. The side button 106 can control a feature and/or component of the computing device 102, such as a volume of a speaker, a power state of the computing device 102, or an operation of a camera of the computing device 102. Other implementations are also possible in which the electrode 104 represents a distinct component that is separate from, but coupled to, the computing device. Although the computing device 102 is depicted as a smartphone in FIG. 1, the computing device 102 can include other types of devices, including those described with respect to FIG. 2.

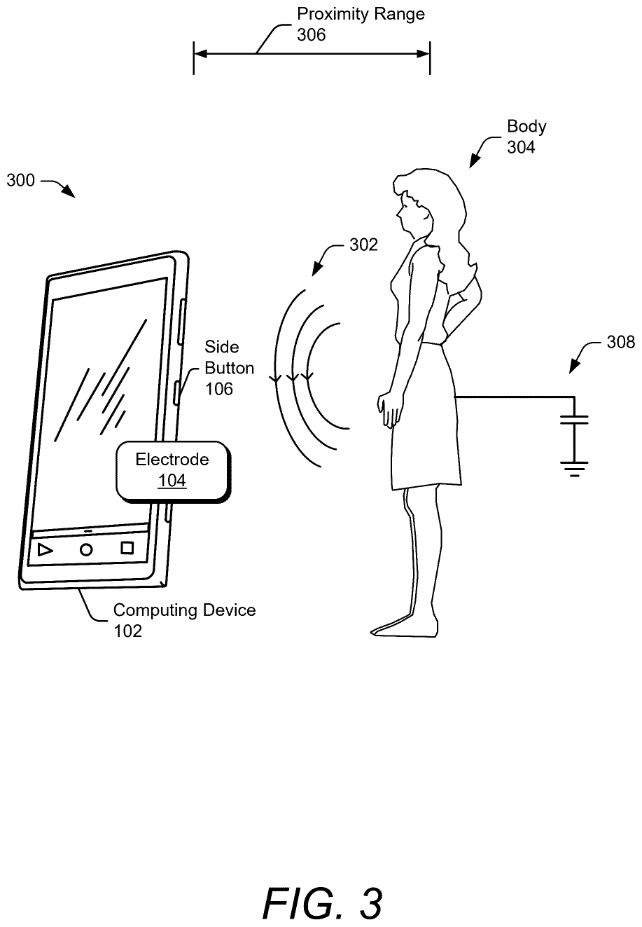

The computing device 102 performs passive sensing 108 using the electrode 104. Through passive sensing 108, the electrode 104 senses an electric field in an external environment (e.g., an electric field that exists outside of the computing device 102), as further described with respect to FIG. 3. Passive sensing 108 refers to an ability of the electrode 104 to detect the electric field without actively emitting energy (e.g., without transmitting a signal). In this way, the electrode 104 can consume significantly less power compared to other sensors that perform active sensing.

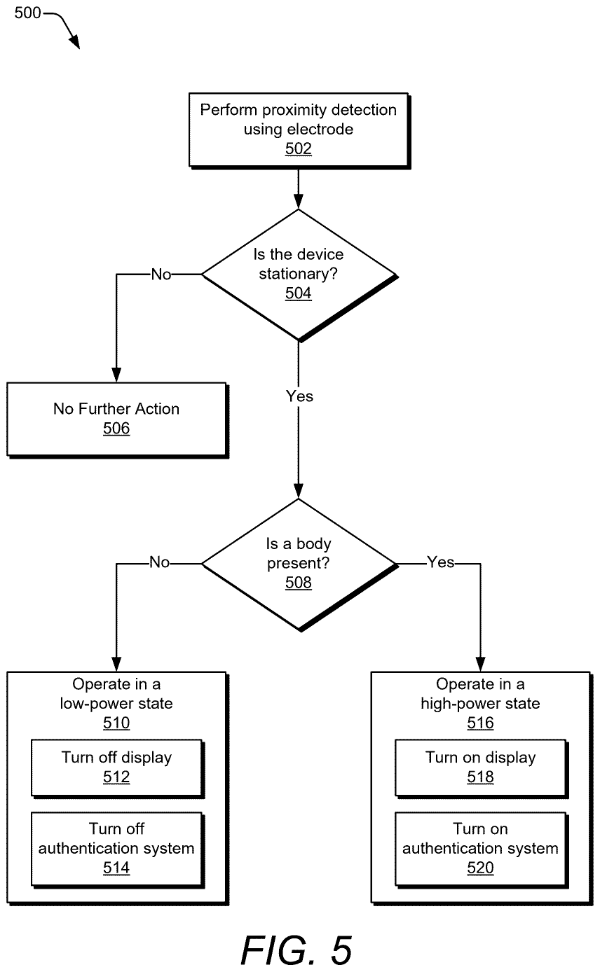

By using the electrode 104 to detect changes in the electric field, the computing device 102 can perform a variety of functions, including proximity detection 110, motion detection 112, sleep-quality assessment 114, electrical-wire detection 116, and/or biometric monitoring 118. With proximity detection 110 and/or motion detection 112, the computing device 102 can appropriately switch between various states to improve the user experience and/or to conserve power, as further described with respect to FIG. 5. With sleep-quality assessment 114, electrical-wire detection 116, and/or biometric monitoring 118, the computing device 102 can provide the user 120 information that they may find useful for improving their health or for completing a home-improvement project.

Proximity detection 110 involves detecting a presence, or an absence, of a user 120. In some cases, proximity detection 110 can also include determining a distance and/or an angle to the user 120. In the environment 100-1, the computing device 102 uses the electrode 104 to detect the presence of the user 120. The user 120 approaches a sink, which is next to the computing device 102, to wash their hands. In this case, the computing device 102 turns on its display based on the detected presence of the user 120. If the user 120 leaves, the computing device 102 can use the electrode 104 to detect the absence of the user 120. In this case, the computing device 102 can turn off the display to conserve power. With the electrode 104, the computing device 102 can realize a sufficient resolution for detecting a person or a portion of a person (e.g., an appendage of the person such as a finger, a hand, or an arm). The electrode 104 can also perform proximity detection 110 across a wide range of distances, including distances as close as a few centimeters to distances of several meters. These distances can include shorter distances at approximately 2, 4, or 5 centimeters and longer distances at approximately 1, 2, 4, 5, or more meters.

Motion detection 112 involves detecting a movement of the user 120. Some example movements can be relatively large, such as the user 120 walking towards or walking away from the computing device 102. Other movements can be relatively small, such as the user 120 moving their fingers in front of the computing device 102. In the environment 100-2, the user 120 reaches to pick up the computing device 102. This reach motion can be detected using the electrode 104. In some cases, motion detection 112 can include detecting and recognizing gestures performed by the user 120.

Sleep-quality assessment 114 is similar to motion detection 112 in that the electrode 104 detects the motion of the user 120 while they are sleeping, as shown in environment 100-3. With the electrode 104, the computing device 102 can determine how often and how much the user 120 moves around in their sleep. A significant amount of movement can indicate poor sleep quality. The computing device 102 utilizes this movement information to assess the quality of the user 120's sleep.

Electrical-wire detection 116 includes detecting the presence of electrical wires, which may be embedded within walls or otherwise obscured from the user 120's view. In this case, the electrode 104 can sense the relatively strong electric field generated by the electrical wires and the computing device 102 can alert the user 120 to the presence of the electrical wires. This feature can be beneficial in situations in which the user 120 is completing a home-improvement project.

Biometric monitoring 118 can include detecting an activity of the user 120's heart. In this example, the electrode 104 can be implemented using an electrocardiogram (ECG) sensor. Using the electrode 104, the computing device 102 can measure the user 120's heart rate and/or detect abnormalities in the rhythm of the user 120's heart (e.g., can detect arrhythmias). The computing device 102 is further described with respect to FIG. 2.

FIG. 2 illustrates an example computing device 102. The computing device 102 is illustrated with various non-limiting example devices including a desktop computer 102-1, a tablet 102-2, a laptop 102-3, a television 102-4, a computing watch 102-5, computing glasses 102-6, a gaming system 102-7, a microwave 102-8, and a vehicle 102-9. Other devices may also be used, such as a home service device, a smart speaker, a smart thermostat, a baby monitor, a Wi-Fi® router, a drone, a trackpad, a drawing pad, a netbook, an e-reader, a home automation and control system, a wall display, and another home appliance. Note that the computing device 102 can be wearable, non-wearable but mobile, or relatively immobile (e.g., desktops and appliances).

The computing device 102 includes at least one electrode 104 and optionally includes at least one motion sensor 202. The electrode 104 can include an impedance circuit that is coupled to a grounding node of the computing device 102. The electrode 104 can detect small changes in an external electric field by sensing a voltage across the impedance circuit. In general, an impedance of the impedance circuit can be set sufficiently high to enable the electrode 104 to realize a target level of sensitivity for proximity detection 110, motion detection 112, sleep-quality assessment 114, electrical-wire detection 116, and/or biometric monitoring.

An operation of the electrode 104 can consume relatively little power (e.g., on the order of milliwatts or less). With this low power consumption, the electrode 104 can operate in a continuous manner while the computing device 102 is powered on. In general, this continuous manner means that the electrode 104 performs multiple measurements of an electric field over time. In some example implementations, the electrode 104 periodically senses the electric field and generates measurements at discrete points in time. Other example implementations are also possible in which the electrode 104 continuously senses the electric field and generates measurements having values for all points in time across a given time interval.

Some implementations of the computing device 102 can include multiple electrodes 104. With multiple electrodes 104, the computing device 102 can make multiple, independent observations regarding the electric field, which can improve the performance of proximity detection 110, motion detection 112, sleep-quality assessment 114, electrical-wire detection 116, and/or biometric monitoring 118. If the multiple electrodes 104 are positioned at different locations within the computing device 102, the measurement provided by the multiple electrodes 104 can be used to perform more complex techniques, such as angle detection.

Example motion sensors 202 include an inertial measurement unit (IMU), an accelerometer, an inclinometer, a gyroscope, a magnetometer, or some combination thereof. In general, the motion sensor 202 detects a motion of the computing device 102 itself. The motion sensor 202 can also be referred to as an on-board motion sensor as it is incorporated within the computing device 102. Data generated by the motion sensor 202 can be used to gate the various passive sensing 108 functions, including the proximity detection 110, the motion detection 112, the sleep-quality assessment 114, the electrical-wire detection 116, and/or the biometric monitoring 118. In particular, the motion sensor 202 enables the computing device 102 to determine if changes in the sensed electric field are due to an object (e.g., the user 120) that is within the external environment or are due to the movement of the computing device 102. In this way, the computing device 102 can reduce false alarms and improve the performance of the various functions associated with passive sensing 108.

The computing device 102 also includes one or more computer processors 204 and at least one computer-readable medium 206, which includes memory media and storage media. The computer-readable medium 206, or alternatively the non-volatile memory within the computer-readable medium 206, may include a non-transitory computer-readable storage medium. In some implementations, the computer-readable medium 206 and/or the non-transitory computer-readable storage medium of the computer-readable medium 206, stores programs, modules, and data structures, or a subset or superset thereof. Applications and/or an operating system (not shown) embodied as computer-readable instructions on the computer-readable medium 206 can be executed by the computer processor 204 to provide some of the functionalities described herein.

The computer-readable medium 206 includes a proximity and/or motion detector 208, which uses information provided by the electrode 104 to perform proximity detection 110 and/or motion detection 112. In example implementations, the proximity and/or motion detector 208 can be implemented using a machine-learned model or another model that performs signal and/or data processing. Generally speaking, the proximity and/or motion detector 208 analyzes the data provided by the electrode 104 to perform proximity detection 110 and/or the motion detection 112. Although not explicitly shown in FIG. 2, the computing device 102 can include other modules for performing sleep-quality assessment 114, electrical-wire detection 116, and/or biometric monitoring 118. These modules can also be implemented using a machine-learned model or another model that performs signal and/or data processing.

The computer-readable medium 206 also includes at least one state manager 210. The state manager 210 can appropriately configure an operational state of the computing device 102 based on the proximity detection 110 and/or based on the motion detection 112. For example, the state manager 210 can cause the computing device 102 to be in different power states and/or can cause different components of the computing device 102 to be powered on or powered off, as further described with respect to FIG. 5.

The computing device 102 can also include a network interface 212 for communicating data over wired, wireless, or optical networks. For example, the network interface 212 may communicate data over a local-area-network (LAN), a wireless local-area-network (WLAN), a personal-area-network (PAN), a wire-area-network (WAN), an intranet, the Internet, a peer-to-peer network, point-to-point network, a mesh network, Bluetooth®, and the like. The computing device 102 may also include a display 214 and/or an authentication system 216. The authentication system 216 can include components such as a camera, a fingerprint sensor, an infrared light, and so forth. The authentication system 216 can utilize one or more techniques known in the art to authentication the user 120, such as fingerprint recognition and/or facial recognition. Operations of the network interface 212, the display 214, and/or the authentication system 216 can be controlled by the state manager 210. An operation of the electrode 104 is further described with respect to FIG. 3.

### Passive Proximity Detection Using an Electrode

FIG. 3 illustrates an example operation of the electrode 104 performing aspects of passive proximity detection. In the environment 300, the electrode 104 can sense the electric field 302, which is associated with an external environment (e.g., an environment that exists outside of the computing device 102). The electric field 302 can also be referred to as a static electric field. The electric field 302 can be generated, at least in part, by stationary charges and currents, which are generally fixed in space.

When a body 304, such as a person (e.g., the user 120), an animal, or an object, is located within a proximity range 306 from the computing device 102, the electrode 104 can sense the change in the electric field 302 as caused by the body 304's presence. This change occurs because the body 304 can carry a static electric field itself, which impacts the charge distribution within the external environment. The body 304 can be represented by a capacitor 308 as it is capable of storing electrical charge. Depending on the situation, the body 304 can increase or decrease an intensity of the electric field 302. The proximity range 306 represents a maximum distance that the electrode 104 can detect a presence and/or a motion of the body 304.

The electric field 302 can be impacted in different ways based on the position of the body 304 and/or the motions made by the body 304. By detecting changes in an intensity of the electric field 302, the computing device 102 can determine that the body 304 is present, can determine a position of the body 304 (e.g., a distance and/or an angle to the body 304), and/or can detect and/or recognize a motion that is performed by the body 304. By detecting the change in the electric field 302, the electrode 104 enables the computing device 102 to perform proximity detection 110 and/or motion detection 112.

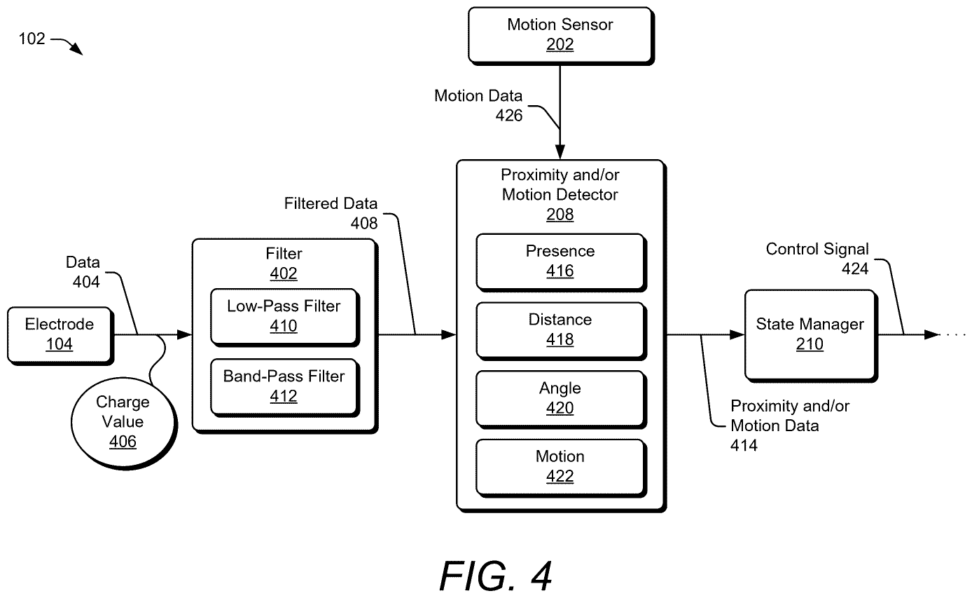

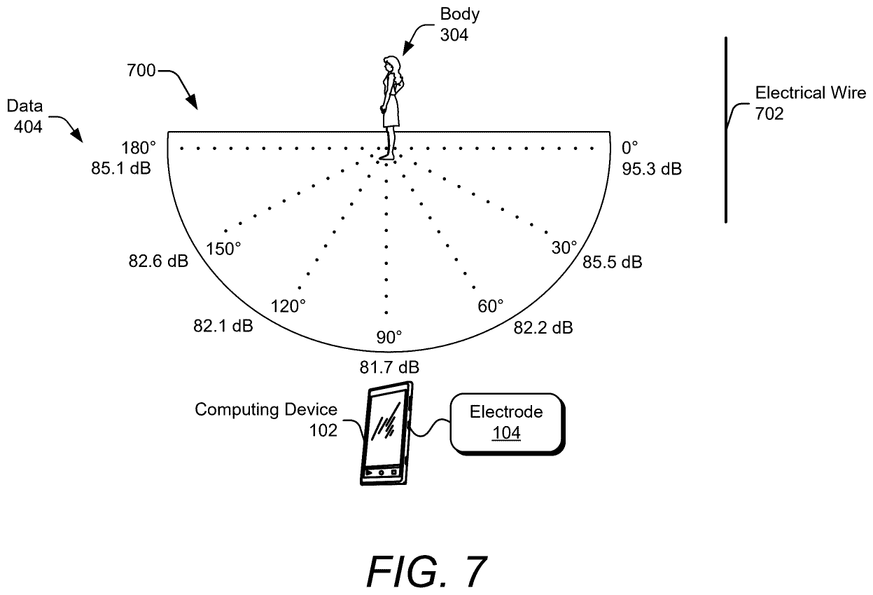

The passive sensing 108 performed using the electrode 104 is omnidirectional (e.g., orientation agnostic). This means that the computing device 102 can perform proximity detection 110 and/or motion detection 112 regardless of which angle the body 304 is positioned relative to the computing device 102. Consider an example situation in which the electrode 104 is integrated within the side button 106, which is shown on a right side of the computing device 102 in FIG. 3. Using the electrode 104, the computing device 102 can detect the presence of the body 304 if an orientation of the computing device 102 causes the side button 106 (e.g., the electrode 104) to be facing towards the body 304, as shown in FIG. 3. The computing device 102 can also detect the presence of the body 304 if an orientation of the computing device 102 causes the side button 106 (e.g., the electrode 104) to be facing away from the body 304. In this case, the body 304 is on an opposite side of the computing device 102 relative to a side of the computing device 102 that includes the side button 106 (e.g., the electrode 104). This orientation agnostic capability of the electrode 104 is further described with respect to FIG. 7. Aspects of proximity detection 110 and/or motion detection 112 are further described with respect to FIG. 4.

FIG. 4 illustrates an example implementation of the computing device 102, which is capable of performing passive proximity detection 110 and/or passive motion detection 112. In the depicted configuration, the computing device 102 includes the electrode 104, at least one optional filter 402, the proximity and/or motion detector 208, and the state manager 210. The filter 402 is coupled between the electrode 104 and the proximity and/or motion detector 208. The proximity and/or motion detector 208 is coupled between the filter 402 and the state manager 210. The computing device 102 can optionally include the motion sensor 202, which is coupled to the proximity and/or motion detector 208 in FIG. 4.

During operation, the electrode 104 senses the electric field 302 to generate data 404. The data 404 can include at least one charge value 406, which represents an intensity of the electric field 302. As explained in FIG. 2, the electrode 104 can include an impedance circuit that is coupled between an electrical conductor of the electrode 104 and a grounding node of the computing device 102. A static voltage can be measured across the impedance circuit and is represented by the charge value 406.

The filter 402 generates filtered data 408 based on the data 404. In example implementations, the filter 402 can include at least one low-pass filter 410 and/or at least one band-pass filter 412. A passband of the filter 402 can be appropriately set for proximity detection 110, motion detection 112, sleep-quality assessment 114, electrical-wire detection 116, and/or biometric monitoring 118. For motion detection 112, the passband can be set between approximately 2 and 3 hertz to detect the body 304 (e.g., the user 120) walking or can be set between approximately 5 and 8 hertz to detect the body 304 running. For electrical-wire detection 116, the passband can include frequencies associated with utility power, such as 60 hertz for the United States of America or 50 Hertz for Europe. With the filter 402, the computing device 102 can attenuate (e.g., substantially remove) noise from the external environment. The passband of the filter 402 can be static or can be dynamically set based on which passive sensing 108 functions are enabled by the computing device 102 and/or the body 304.

The proximity and/or motion detector 208 generates proximity and/or motion data 414 based on the filtered data 408. In various implementations, the proximity and/or motion detector 208 can determine a presence 416 (or absence) of the body 304, measure a distance 418 between the computing device 102 and the body 304, can determine an angle 420 to the body 304 (e.g., can determine an angular position of the body 304 relative to the computing device 102), and/or can detect motion 422 of the body 304 (or the absence of motion 422). The determined presence 416, distance 418, angle 420, and/or motion 422 can be passed to the state manager 210 and/or included as part of the proximity and/or motion data 414.

Various techniques can be used to determine the distance 418. In a first example, the distance 418 can be determined based on the proximity range 306 of the electrode 104. In this case, a sensitivity of the electrode 104 can be adjusted to change the proximity range 306. By sensing the electric field 302 using different sensitivity levels, the proximity and/or motion detector 208 can determine an approximate distance 418 of the body 304. Other techniques are also possible in which the distance 418 is determined based on the amount of a magnitude of the charge value 406 or based on an amount of variation within the magnitude of the charge value 406. In many situations, larger magnitudes and/or larger variations in the magnitude can indicate that the body 304 is closer to the computing device 102 while smaller magnitudes and/or smaller variations in the magnitude can indicate that the body 304 is farther away from the computing device 102. Other techniques that utilize a machine-learned model are also possible.

To determine the angle 420, the proximity and/or motion detector 208 can compare the data 404 between two electrodes 104 that are positioned at different locations. The differences in location enable the proximity and/or motion detector 208 to determine the angle 420 through techniques such as triangulation. Other techniques that utilize a machine-learned model are also possible. With a machine-learned model, it may be possible to determine the angle 420 based on the data 404 from a single electrode 104.

Additional information can be provided to the proximity and/or motion detector 208 to assist with determining the angle 420. For example, an orientation of the computing device 102 can be provided to the proximity and/or motion detector 208. This can be used for situations in which the electrode 104 senses the electric field 302 while the body 304 (e.g., the user 120) changes an orientation of the computing device 102 for electrical-wire detection 116.

The state manager 210 controls a state of the computing device 102. In this example, the state manager 210 generates a control signal 424 based on the proximity and/or motion data 414. In some examples, the control signal 424 causes the computing device 102 to switch between different power states when there is a change in the proximity and/or motion data 414. For example, the state manager 210 can cause the computing device 102 to be in a high-power state if the proximity and/or motion data 414 indicates that the body 304 is present and/or indicates that the body 304 is moving. Alternatively, the state manager 210 can cause the computing device 102 to be in a low-power state if the proximity and/or motion data 426 indicates that the body 304 is absence and/or indicates that the body 304 is relatively stationary. Other states, such as intermediary power states, are also possible and can be controlled based on the distance 418 and/or the angle 420.

Still other states may not necessarily be related to power consumption and can instead involve an operating state of a particular component within the computing device 102. Example states can control a communication mode and/or an operating frequency of a transceiver of the network interface 212, control a brightness of the display 214, control a volume of a speaker of the computing device 102, or control an on/off state of a microphone of the computing device 102 on or off. Another example state can include a warm-up state (or an initialization) state of a particular component or system within the computing device 102. In this case, the state manager 210 can cause the component to transition from a powered-off state to the warm-up state based on the body 304 being detected. This proactive state transition can ensure the component has completed an initialization process and has entered an active state by a time the body 304 is close enough to the computing device 102 to interact with the computing device 102. This level of responsiveness, as afforded through proximity detection 110, can improve the user experience by removing delays associated with components of the device 102 going through a warm-up sequence. Furthermore, power can be conserved while the body 304 is absent.

In some implementations, the motion sensor 202 controls an operation of the proximity and/or motion detector 208. In this example, the motion sensor 202 generates motion data 426, which indicates if the computing device 102 is moving or is relatively stationary. If the motion data 426 indicates that the computing device 102 is relatively stationary, the proximity and/or motion detector 208 can perform the proximity detection 110 and/or motion detection 112. Otherwise, if the motion data 426 indicates that the computing device 102 is moving, the proximity and/or motion detector 208 can temporarily stop performing the proximity detection 110 and/or the motion detection 112. In this case, the proximity and/or motion detector 208 can discard the data 404 or the filtered data 408 associated with a time that the computing device 102 is determined to be in motion according to the motion data 426. This ensures that the proximity and/or motion data 414 corresponds to elements in the environment (e.g., objects and/or people, including the body 304) instead of a movement of the computing device 102. In this manner, a false alarm rate of the proximity and/or motion detector 208 can be decreased. An example operation of the computing device 102 is further described with respect to FIG. 5.

FIG. 5 illustrates an example scheme 500 that utilizes passive proximity detection 110 to control an operational state of the computing device 102. At 502, the computing device 102 performs proximity detection 110 using the electrode 104, as explained above with respect to FIG. 4. At 504, the computing device 102 uses the motion sensor 202 to determine if the computing device 102 is stationary (or substantially stationary). The computing device 102 can determine that it is relatively stationary based on the motion data 426 indicating that movement of the computing device 102 is less than a threshold. If the computing device 102 is not stationary, no further action is taken at 506 and the computing device 102 remains in its current state. If the computing device 102 is stationary at 504, the process can continue to 508.

At 508, the computing device 102 determines if the body 304 is present. For example, the proximity and/or motion detector 208 detects the presence 416 and/or detects the motion 422 of the body 304. If the body 304 is absent (e.g., outside of the proximity range 306), the state manager 210 causes the computing device 102 to operate in a low-power state, as indicated at 510. The low-power state can correspond to the computing device 102 consuming a first amount of power. In some cases, the low-power state can cause the computing device 102 to turn off the display 214 and/or turn off the authentication system 216, as indicated at 512 and 514, respectively. That is, the low-power state may be a state in which one or both of the display 214 and the authentication system 216 are turned off.

Alternatively, if the body 304 is present (e.g., within the proximity range 306), the state manager 210 causes the computing device 102 to operate in a high-power state, as indicated at 516. The high-power state can correspond to the computing device 102 consuming a second amount of power, which is larger than the first amount of power. In some cases, the high-power state can cause the computing device 102 to turn on the display 214 and/or turn on the authentication system 216, as indicated at 518 and 520, respectively. That is, the high-power state may be a state in which one or both of the display 214 and the authentication system 216 are turned off.

Other implementations are also possible in which the step at 508 includes determining if the body 304 is moving. By dynamically changing the state of the computing device 102 based on the passive sensing 108, the computing device 102 can conserve power while ensuring responsiveness and/or improving the user experience.

While in the low-power state or the high-power state, the electrode 104 can continue sensing the electric field 302 and generating the data 404. In other words, the computing device 102 can continue performing passive proximity detection 110 at 502. The data 404 generated by the electrode 104 is further described with respect to FIG. 5.

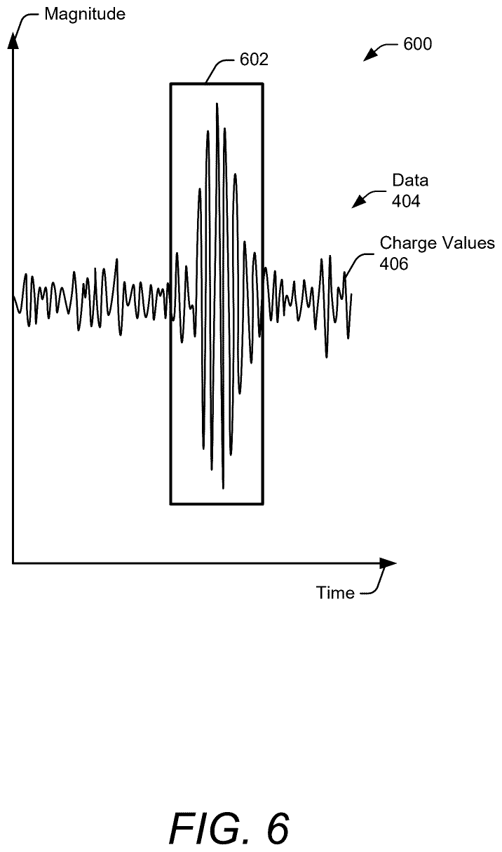

FIG. 6 illustrates example data 404 generated by the electrode 104 for passive sensing 108. A graph 600 depicts a magnitude of the charge values 406 over time. At 602, the body 304 is present within the proximity range 306 and/or moves within the proximity range 306. For example, the body 304 (e.g., the user 120) can be walking towards the computing device 102 and enters the proximity range 306 at 602. In another example, the body 304 is present within the proximity range 306 and performs a gesture using a human appendage (e.g., the user 120 performs a gesture using their hand) at 602. The presence 416 and/or motion 422 of the body 304 causes the magnitude of the charge values 406 to vary significantly at 602.

The proximity and/or motion detector 208 can analyze changes in the magnitude of the charge values 406 over time to detect the presence 416 and/or the motion 422 of the body 304. In some instances, the change can be relative to a previous state or relative to a previous trend in the data 404. The previous state can refer to a portion of the data 404 during which the body 304 is absent (e.g., is outside of the proximity range 306) and/or a portion of the data 404 during which the body 304 is relatively stationary. The proximity and/or motion detector 208 can utilize a threshold and/or can employ machine learning to detect the change in the data 404 at 602 and associate the change with the presence 416 and/or the motion 422 of the body 304.

The term “significantly” can mean that the values of the magnitude can change by 20% or more relative to a previous value (e.g., relative to an average of a set of previous values). Additionally or alternatively, a slope of the data 404 can vary significantly. Sometimes the slope of the data 404 can change signs (e.g., from a positive slope to a negative slope, or vice versa). A magnitude of the slope of the data 404 can sometimes change by approximately 10% or more. The specific values of the change in magnitude or slope that may be determined to be “significant” may be determined empirically.

FIG. 7 depicts an example plot 700 of the data 404 over angle of incidence for passive sensing 108. In examples where the body 304 (e.g., the user 120) or a movement of the body 304 may be detected in an environment by one or more electrodes 104 on the computing device 102, the body 304 or the movement of the body 304 may be at an angle relative to the orientation of the one or more electrodes 104 (e.g., relative to an orientation of the computing device 102). The plot 700 shows example magnitudes of the data 404 (in decibels (dB)), which is detected across different angles of incidences (in degrees) between the body 304 and the computing device 102.

In this example, there is not a noticeable degradation in performance based on angle of incidence. The data 404 across angles of incidences of approximately 0 to 180 degrees, for instance, are relatively similar. This means that the passive sensing 108 performed using the electrode 104 is agnostic to orientation. In other words, the computing device 102 can perform passive sensing 108 using the electrode 104 regardless of an angular position of the body 304 (e.g., regardless of whether the body 304 is on a same side of the computing device 102 as the electrode 104 or is on an opposite side of the computing device 102 relative to the electrode 104).

At the angle of incidence of 0 degrees, the computing device 102 is proximate to one or more electrical wires 702, which can be located behind a wall. The magnitude of the data 404 at this angle of incidence is higher due to current that is conducted along the electrical wire 702. As can be seen in FIG. 7, the computing device 102 can use this data 404 to detect the presence of the electrical wire 702. The computing device 102 can use a machine-learned model or a data-processing model to recognize the presence of the electrical wire 702 based on the magnitude of the data 404. Although many of the examples described herein are described with respect to detecting a presence 416 and/or motion 422 of the body 304, similar techniques can be applied to sensing the presence 416 and/or motion 422 of other objects, other people, and/or other living things. Example objects include those that have a static electric field (e.g., a static charge).

### Example Method

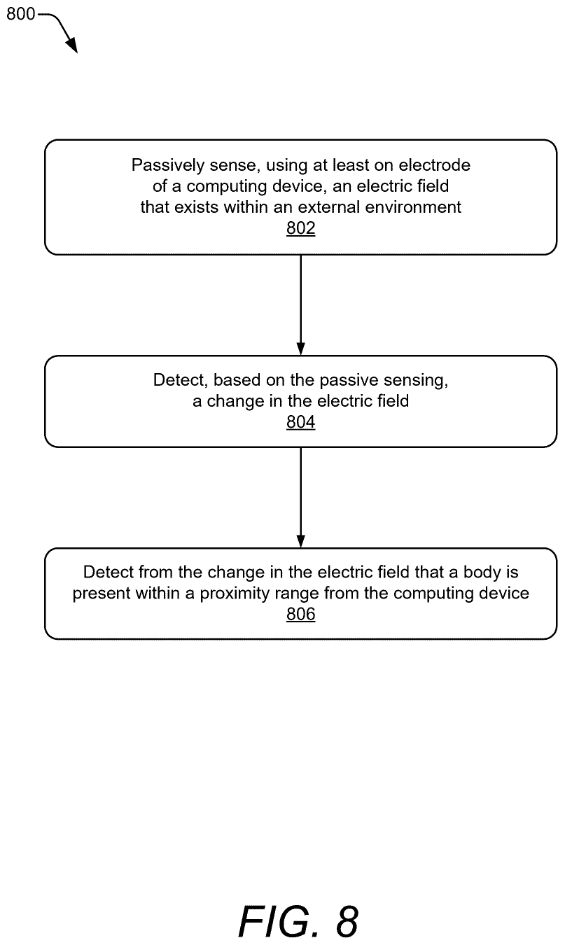

FIG. 8 depicts an example method 800 for implementing aspects of passive proximity detection using an electrode. Method 800 is shown as a set of operations (or acts) performed but not necessarily limited to the order or combinations in which the operations are shown herein. Further, any of one or more of the operations may be repeated, combined, reorganized, or linked to provide a wide array of additional and/or alternate methods. In portions of the following discussion, reference may be made to the environment 100 of FIG. 1, and entities detailed in FIGS. 2 and 4, reference to which is made for example only. The techniques are not limited to performance by one entity or multiple entities operating on one device.

At 802, an electric field that exists within an external environment is passively sensed using at least one electrode of a computing device. For example, the computing device 102 performs passive sensing 108 using at least one electrode 104, as shown in FIG. 1. The electrode 104 senses the electric field 302 within an external environment, as shown in FIG. 3. By sensing the electric field 302, the electrode 104 generates data 404, which can include one or more charge values 406. Each charge value 406 represents an intensity of the electric field 302. The electrode 104 senses the electric field 302 without emitting energy (e.g., without transmitting a signal). In this way, the computing device 102 can continuously perform passive sensing 108 without significantly impacting power resources.

In some implementations, the electrode 104 is implemented as an electrocardiogram sensor. The electrode 104 can be incorporated into a side button 106 of the computing device 102. The at least one electrode 104 can include at least two electrodes 104 (e.g., at least two electrocardiogram sensors). The at least two electrodes 104 can be incorporated into the side button 106 or positioned further apart. In this case, both electrodes 104 can sense the electric field 302 within the external environment. The additional data 404 generated by a second electrode 104 can be used to further increase the performance of passive proximity detection 110 by improving the accuracy and/or by enabling more advanced techniques to be applied.

At 804, a change in the electric field is detected based on the passive sensing. For example, the computing device 102 detects a change in the electric field 302 based on the passive sensing 108. More specifically, the proximity and/or motion detector 208 detects a change in the data 404 generated by the electrode 104. The change can represent a change in a magnitude of the charge value 406 or a change in a slope of the magnitude of the charge value 406.

At 806, a body that is present within a proximity range from computing device is detected from the change in the electric field. For example, the computing device 102 detects that a body 304 is within a proximity range 306 from the computing device 102 based on the change in the electric field 302, as shown in FIG. 3. The proximity range 306 represents a maximum range at which the electrode 104 can sense the presence 416 of the body 304. In some cases, the computing device 102 can change its state based on the determined presence 416 of the body 304, as described with respect to FIG. 5.

### Example Computing System

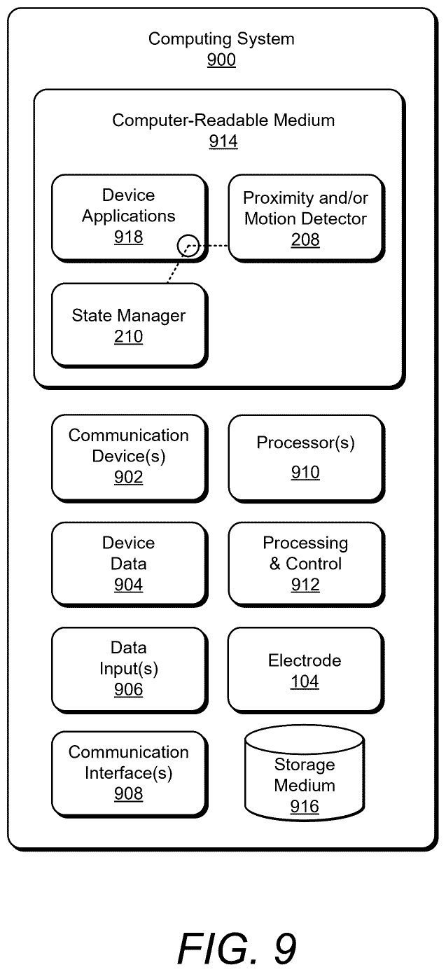

FIG. 9 illustrates various components of an example computing system 900 that can be implemented as any type of client, server, and/or computing device as described with reference to the previous FIGS. 2 and 4 to implement aspects of passive proximity detection using an electrode. The computing system 900 includes at least one electrode 104. Although not explicitly shown, the computing system 900 can also include the motion sensor 202 shown in FIG. 2.

The computing system 900 also includes communication devices 902 that enable wired and/or wireless communication of device data 904 (e.g., received data, data that is being received, data scheduled for broadcast, or data packets of the data). The device data 904 or other device content can include configuration settings of the device, media content stored on the device, and/or information associated with a user of the device. Media content stored on the computing system 900 can include any type of audio, video, and/or image data. The computing system 900 includes one or more data inputs 906 via which any type of data, media content, and/or inputs can be received, such as human utterances, user-selectable inputs (explicit or implicit), messages, music, television media content, recorded video content, and any other type of audio, video, and/or image data received from any content and/or data source.

The computing system 900 additionally includes communication interfaces 908, which can be implemented as any one or more of a serial and/or parallel interface, a wireless interface, any type of network interface, a modem, and as any other type of communication interface. The communication interfaces 908 provide a connection and/or communication links between the computing system 900 and a communication network by which other electronic, computing, and communication devices communicate data with the computing system 900.

The computing system 900 includes one or more processors 910 (e.g., any of microprocessors, controllers, and the like), which process various computer-executable instructions to control the operation of the computing system 900. Alternatively or in addition, the computing system 900 can be implemented with any one or combination of hardware, firmware, or fixed logic circuitry that is implemented in connection with processing and control circuits which are generally identified at 912. Although not shown, the computing system 900 can include a system bus or data transfer system that couples the various components within the device. A system bus can include any one or combination of different bus structures, such as a memory bus or memory controller, a peripheral bus, a universal serial bus, and/or a processor or local bus that utilizes any of a variety of bus architectures.

The computing system 900 also includes a computer-readable medium 914, such as one or more memory devices that enable persistent and/or non-transitory data storage (i.e., in contrast to mere signal transmission), examples of which include random access memory (RAM), non-volatile memory (e.g., any one or more of a read-only memory (ROM), flash memory, EPROM, EEPROM, etc.), and a disk storage device. The disk storage device may be implemented as any type of magnetic or optical storage device, such as a hard disk drive, a recordable and/or rewriteable compact disc (CD), any type of a digital versatile disc (DVD), and the like. The computing system 900 can also include a mass storage medium device (storage medium) 916.

The computer-readable medium 914 provides data storage mechanisms to store the device data 904, as well as various device applications 918 and any other types of information and/or data related to operational aspects of the computing system 900. For example, an operating system 920 can be maintained as a computer application with the computer-readable medium 914 and executed on the processors 910. The device applications 918 may include a device manager, such as any form of a control application, software application, signal-processing and control module, code that is native to a particular device, a hardware abstraction layer for a particular device, and so on.

The device applications 918 also include any system components, engines, or managers to perform aspects of passive sensing 108, including proximity detection 110, motion detection 112, sleep-quality assessment 114, electrical-wire detection 116, biometric monitoring 118, or combinations thereof. In this example, the device applications 918 include the proximity and/or motion detector 208 of FIG. 2 for performing proximity detection 110 and/or motion detection 112. The device applications 918 can also optionally include the state manager 210 of FIG. 2.

Throughout this disclosure, examples are described where a computing system 900 (e.g., the computing device 102) may analyze information associated with the user 120, for example the data 404 mentioned with respect to FIG. 4. The data 404 can be associated with a location of the user 120 relative to the computing device 102, a motion of the user 120, and/or biometrics of the user 120. Further to the descriptions above, a user 120 may be provided with controls allowing the user 120 to make an election as to both if and when systems, programs, and/or features described herein may enable collection of information (e.g., information associated with proximity detection 110, motion detection 112, sleep-quality assessment 114, electrical-wire detection 116, and/or biometric monitoring 118), and if the user 120 is sent content or communications from a server. The computing system 900 can be configured to only use the information after then computing system 900 receives explicit permission from the user 120 to use the data. For example, in situations where the computing system 900 uses the electrode 104 to perform passive sensing 108, individual users 120 may be provided with an opportunity to provide input to control whether programs or features of the computing system 900 can collect and make use of the data 404 generated by the electrode 104. Further, individual users 120 may have constant control over what programs can or cannot do with the information.

In addition, information collected may be pre-treated in one or more ways before it is transferred, stored, or otherwise used, so that personally-identifiable information is removed. For example, before the computing system 900 shares data with another device, a user 120's identity may be treated so that no personally identifiable information can be determined for the user 120. Thus, the user 120 may have control over whether information is collected about the user 120 and the user 120's device, and how such information, if collected, may be used by the computing system 900 and/or a remote computing system.

### Conclusion

Although techniques using, and apparatuses including, passive proximity detection using an electrode have been described in language specific to features and/or methods, it is to be understood that the subject of the appended claims is not necessarily limited to the specific features or methods described. Rather, the specific features and methods are disclosed as example implementations of passive proximity detection using an electrode.

Some Examples are described below.

Example 1: A method performed by a computing device, the method comprising:
passively sensing, using at least one electrode of the computing device, an electric field that exists within an external environment;
detecting, based on the passive sensing, a change in the electric field; and
detecting from the change in the electric field that a body is present within a proximity range from the computing device.

Example 2: The method of example 1, further comprising:
measuring, using the at least one electrode, a heart rate from a human appendage placed over a portion of the computing device that includes the at least one electrode.

Example 3: The method of example 1 or 2, further comprising:
operating the computing device in a low-power state prior to the detecting that the body is present; and
operating the computing device in a high-power state responsive to the detecting that the body is present.

Example 4: The method of example 3, further comprising:
detecting, based on the passive sensing and during a second time, a second change in the electric field;
determining, based on the detecting of the second change during the second time, that the body is outside of the proximity range; and
operating the computing device in the low-power state responsive to the determining that the body is outside of the proximity range.

Example 5: The method of any previous example, further comprising:
determining, using at least one motion sensor of the computing device, that the computing device is substantially stationary,
wherein the determining that the body is within the proximity range is based on the detecting of the change in the electric field and is based on the determining that the computing device is substantially stationary.

Example 6: The method of any previous example, wherein:
the passive sensing comprises:
   generating, by the at least one electrode, a first charge value representing an intensity of the electric field at a first time interval; and
   generating, by the at least one electrode, a second charge value representing the intensity of the electric field at a second time interval; and
the detecting the change in the electric field comprises detecting the change in the electric field based on a comparison of the first charge value with the second charge value.

Example 7: The method of example 6, further comprising:
determining a distance between the body and the computing device based on the second charge value.

Example 8: The method of any previous example, wherein:
the passive sensing comprises:
   generating, by a first electrode of the at least one electrode, a third charge value representing an intensity of the electric field at a third time; and
   generating, by a second electrode of the at least one electrode, a fourth charge value representing the intensity of the electric field at a fourth time; and
the detecting the change in the electric field comprises detecting the change in the electric field based on a comparison of the third charge value with the fourth charge value.

Example 9: The method of any previous example, wherein:
the at least one electrode is positioned on a first side of the computing device; and
a second side of the computing device that is opposite the first side is oriented towards the body.

Example 10: The method of any previous example, wherein:
the body is associated with a sleeping person; and
the method further comprises:
   detecting a motion of the person based on the change in the electric field; and
   estimating a quality of the person's sleep based on the detecting of the motion.

Example 11: The method of any previous example, further comprising:
detecting a presence of an electrical wire based on the passive sensing.

Example 12: The method of any previous claim, wherein the passive sensing comprises passively sensing the electric field without emitting energy within the external environment.

Example 13: An apparatus comprising:
at least one electrode,
the apparatus configured to perform, using the at least one electrode, any one of the methods of examples 1 to 12.

Example 14: The device of example 13, wherein:
the device comprises a button that is positioned on one side of the device; and
the at least one electrode is integrated within the button.

Example 15: The device of example 13 or 14, further comprising an electrocardiogram sensor, the electrocardiogram sensor including the at least one electrode.

Example 16: The device of any one of examples 13 to 15, wherein the at least one electrode comprises at least two electrodes.

Example 17: The device of any one of examples 13 to 16, wherein the at least one electrode is configured to operate in a continuous manner while the apparatus is powered on.

Example 18: A computer-readable storage medium comprising instructions that, responsive to execution by a processor, cause a computing device to perform any one of the methods of examples 1 to 12.

Example 19: A method for proximity sensing, the method comprising:
receiving, by one or more processors, a first charge value, the first charge value based on first electrode data produced by a first electrode at a first time;
receiving, by the one or more processors, a second charge value, the second charge value based on second electrode data produced by a second electrode at a second time;
comparing, by the one or more processors, the first charge value and the second charge value; and
determining, by the one or more processors and based on the comparison, whether a motion has been detected.

Example 20: The method of example 19, wherein the first and second electrodes are the same electrode.

Example 21: The method of example 19 or 20, further comprising:
determining, by the one or more processors and based on the comparison, whether a charge source emitter is emitting a charge distribution; and
responsive to the determination that the charge source emitter is emitting a charge distribution, determining, by the one or more processors, at least one of a direction or a distance of the charge source emitter relative to a mobile electronic device.

Example 22: A mobile electronic device, the mobile electronic device comprising:
one or more electrodes, the one or more electrodes configured to produce electrode data based on an electric charge sensed by the one or more electrodes;
one or more processors; and
a memory storing instructions that, when executed by the one or more processors, cause the one or more processors to:
   receive a first charge value, the first charge value based on first electrode data produced by the one or more electrodes at a first time;
   receive a second charge value, the second charge value based on second electrode data produced by the one or more electrodes at a second time;
   compare the first charge value and the second charge value; and
   determine, based on the comparison, whether a motion has been detected.

Example 23: The mobile electronic device of example 22, wherein the one or more electrodes are configured to function as electrocardiogram (ECG) sensors.

Example 24: The mobile electronic device of example 22 or 23, wherein:
the electric charge sensed by the one or more electrodes is caused by a corresponding electric field (E-field);
the first electrode data is based on a first E-field value of the E-field at the first time; and
the second electrode data is based on a second E-field value of the E-field at the second time.

Example 25: The mobile electronic device of example 22, 23, or 24, wherein the instructions further cause the one or more processors to:
determine, based on the comparison, whether a charge source emitter is emitting a charge distribution; and
   responsive to a determination that the charge source emitter is emitting a charge distribution, determine at least one of a direction or a distance of the charge source emitter relative to the mobile electronic device.

1. A method performed by a computing device, the method comprising:
passively sensing, during a first time and using at least one electrode of the computing device, an electric field that exists within an external environment;
detecting, based on the passive sensing, a change in the electric field;
detecting from the change in the electric field that a body is present within a proximity range from the computing device; and
measuring, during a second time and using the at least one electrode, a heart rate from a human appendage placed over a portion of the computing device that includes the at least one electrode.

2. The method of claim 1,
wherein the at least one electrode is part of an electrocardiogram sensor of the computing device.

3. The method of claim 1, further comprising:
operating the computing device in a low-power state prior to the detecting that the body is present; and
operating the computing device in a high-power state responsive to the detecting that the body is present.

4. The method of claim 3, further comprising:
detecting, based on the passive sensing and during a third time, a second change in the electric field;
determining, based on the detecting of the second change during the third time, that the body is outside of the proximity range; and
operating the computing device in the low-power state responsive to the determining that the body is outside of the proximity range.

5. The method of claim 1, further comprising:
determining, using at least one motion sensor of the computing device, that the computing device is substantially stationary,
wherein the determining that the body is within the proximity range is based on the detecting of the change in the electric field and is based on the determining that the computing device is substantially stationary.

6. The method of claim 1, wherein:
the passive sensing comprises:
generating, by the at least one electrode, a first charge value representing an intensity of the electric field at a first time interval; and
generating, by the at least one electrode, a second charge value representing the intensity of the electric field at a second time interval; and
the detecting the change in the electric field comprises detecting the change in the electric field based on a comparison of the first charge value with the second charge value.

7. The method of claim 6, further comprising:
determining a distance between the body and the computing device based on the second charge value.

8. The method of claim 1, wherein:
the passive sensing comprises:
generating, by a first electrode of the at least one electrode, a third charge value representing an intensity of the electric field at a third time interval; and
generating, by a second electrode of the at least one electrode, a fourth charge value representing the intensity of the electric field at a fourth time interval; and
the detecting the change in the electric field comprises detecting the change in the electric field based on a comparison of the third charge value with the fourth charge value.

9. The method of claim 1, wherein:
the at least one electrode is positioned on a first side of the computing device; and
a second side of the computing device that is opposite the first side is oriented towards the body.

10. The method of claim 1, wherein:
the body is associated with a sleeping person; and
the method further comprises:
detecting a motion of the person based on the change in the electric field; and
estimating a quality of the person's sleep based on the detecting of the motion.

11. The method of any previous claim 1, further comprising:
detecting a presence of an electrical wire based on the passive sensing.

12. The method of claim 1, wherein the passive sensing comprises passively sensing the electric field without emitting energy within the external environment via the at least one electrode.

13. A device comprising:
at least one electrode configured to:
passively sense, during a first time, an electric field that exists within an external environment;
generate, during the first time, data that is representative of the passively sensed electric field; and
measure, during a second time, a heart rate from a human appendage placed over a portion of the device that includes the at least one electrode; and
a processor that is coupled to the at least one electrode, the processor configured to:
detect, based on the data, a change in the electric field; and
detect from the change in the electric field that a body is present within a proximity range from the device.

14. The device of claim 13, wherein:
the device comprises a button that is positioned on one side of the device; and
the at least one electrode is integrated within the button.

15. The device of claim 13, further comprising an electrocardiogram sensor, the electrocardiogram sensor including the at least one electrode.

16. The device of claim 13, wherein the at least one electrode comprises at least two electrodes.

17. The device of claim 13, wherein the at least one electrode is configured to operate in a continuous manner while the device is powered on.

18. A computer-readable storage medium comprising instructions that, responsive to execution by a processor, cause the processor to:
receive first data from at least one electrode, the first data representing measurements of an electric field that is passively sensed by the at least one electrode during a first time, the at least one electrode comprising an electrocardiogram sensor;
detect, based on the first data, a change in the electric field; and
detect from the change in the electric field that a body is present within a proximity range from the at least one electrode.

19. The computer-readable storage medium of claim 18, wherein the instructions cause the processor to:
cause a computing device to operate in a low-power state prior to the detecting that the body is present, the computing device comprising the at least one electrode; and
cause the computing device to operate in a high-power state responsive to detecting that the body is present.

20. The computer-readable storage medium of claim 19, wherein the instructions cause the processor to:
receive second data from the at least one electrode, the second data representing measurements of the electric field that is passively sensed by the at least one electrode during a second time;
detect, based on the second data, a second change in the electric field;
determine, based on the detected second change, that the body is outside of the proximity range; and
cause the computing device to operate in the low-power state responsive to the determining that the body is outside of the proximity range.
